(19) United States
(12) Patent Application Publication
(10) Pub. No.: US 2007/0264661 A1
(43) Pub. Date: Nov. 15, 2007

(54) METHOD FOR RAPID IDENTIFICATION OF MOLECULES WITH FUNCTIONAL ACTIVITY TOWARDS BIOMOLECULAR TARGETS

(75) Inventors: Steven A. Hofstadler, Vista, CA (US); Lendell L. Cummins, San Diego, CA (US); Kristin A. Sannes-Lowery, Vista, CA (US)

Correspondence Address: Pepper Hamilton LLP, 500 Grant Street, 50th Floor, Pittsburgh, PA, 15219, US

(73) Assignee: Isis Pharmaceuticals, Inc., Carlsbad, CA (US)

(21) Appl. No.: 11/774,463

(22) Filed: Jul. 6, 2007

Related U.S. Application Data
(63) Continuation of application No. 10/922,739, filed on Aug. 20, 2004.
(60) Provisional application No. 60/496,951, filed on Aug. 20, 2003.

(51) Int. Cl. C12Q 1/68; G01N 33/53; G01N 33/573

(52) U.S. Cl. 435/6; 435/7.1; 435/7.4

(57) ABSTRACT

The present invention provides mass spectrometry-based methods for high throughput identification of molecules with functional activities such as nuclease, protease, reductase, kinase, phosphatase, or transferase activities towards biomolecular targets. These methods are useful for screening biological samples such as extracts, broths, lysates and natural product mixtures and provide valuable insights into biomolecular interactions of various biological ligands with biomolecular targets.

### CROSS REFERENCE TO RELATED APPLICATIONS

The present application is a continuation of U.S. patent application Ser. No. 10/922,739, filed Aug. 20, 2004; which claims priority benefit of U.S. Provisional Application 60/496,951, filed Aug. 20, 2003, each of which is hereby incorporated by reference in its entirety.

### SEQUENCE LISTING

The present application is being filed along with a Sequence Listing in electronic format. The Sequence Listing is provided as a file entitled MSIBIS0004USC1SEQ.TXT, created on Jul. 6, 2007 which is 4 Kb in size. The information in the electronic format of the sequence listing is incorporated herein by reference in its entirety.

### FIELD OF THE INVENTION

The present invention is directed to methods for the use of mass spectrometry for the identification of molecules with functional activity toward biomolecular targets. These methods can be performed in parallel with methods for the use of mass spectrometry for screening biochemical samples such as extracts, lysates or broths for individual compounds that bind to a selected target.

### BACKGROUND OF THE INVENTION

The characterization of biologically active fractions from collections of natural products presents many challenges. The many issues include detection of active compounds present at low concentrations in a background of other active species and “false” positives resulting from the summed activity of many weakly active compounds. Historically, mixtures of similar compounds are separated using chromatographic methods prior to screening.

Electrospray ionization mass spectrometry (ESI-MS) can be used as a rapid screening method for identification of active compounds from crude mixtures. ESI-MS allows the simultaneous analysis of mixtures of compounds based on their unique molecular masses. In addition, active compounds can be identified directly from their noncovalent complexes with the target molecules. Control targets can be included in the screening mixture to provide a measure of binding specificity. ESI-MS has high sensitivity and resolving power that facilitates the analysis of trace levels of complex mixtures and such analysis can be implemented in a high-throughput modality with the appropriate robotic interfaces.

At the core of this approach is the use of electrospray ionization (ESI) Fourier transform ion cyclotron resonance (FTICR) mass spectrometry (MS) to characterize noncovalent complexes comprised of a molecular target such as structured RNA or protein and a small molecule ligand. Mass measurements of the intact complex, exact mass measurements of the affinity-selected ligand and subsequent tandem MS measurements are used to gain insight into the composition and structure of the binding species.

Targeting structured RNA presents new opportunities for drug discovery. Structured RNA plays multiple essential roles in protein production. In addition to the role of mRNA carrying the linear coded message for translation into proteins, structured regions of certain mRNAs control the level of protein production by binding to proteins and binding of small molecules to these structures may actually increase protein production.

Many viral and cellular mRNAs contain a structured 5′-untranslated region that may be of interest as a drug target. This region, known as the internal ribosome entry site (IRES) enables binding to a ribosome and initiation of protein translation without the presence of a traditional 5′ cap.

One of the most studied and important structured RNA targets is the prokaryotic ribosomal RNA. The aminoglycoside class of antibiotics causes misreading of the genetic code by binding to the 16S RNA subunit of the prokaryotic ribosome. Binding occurs in a structured region of the 16S RNA known as the A-site.

Disclosed and claimed in U.S. Pat. Nos. 6,428,956, 6,656,690 and 6,770,486 (which are commonly owned and incorporated herein by reference in entirety) are methods for rapid determination of the binding of compounds to biomolecular targets in a massively parallel fashion using ESI-FTICR MS.

Also of interest in natural product fractions is the presence of molecules that bind to the biomolecular target of interest and possess some type of functional activity that causes a modification of the target and/or the binding molecule. These changes may include the addition or removal of a moiety to the target and/or the binding molecule, or cleavage of the target by enzymatic activity of the binding molecule. Examples of functional activities include but are not limited to: nuclease, protease, reductase, kinase, phosphatase, or transferase.

There remains an unmet need for methods for high-throughput characterization of molecules with functional activity from collections of natural products. The present invention satisfies this need.

### SUMMARY OF THE INVENTION

The present invention is directed to a method for identification of a molecule with functional activity towards a biomolecular target in a biological sample comprising contacting a biomolecular target and a control biomolecular target with a biological sample, fractionating the biological sample to obtain a plurality of fractions, and analyzing one or more members of the plurality of fractions by mass spectrometry, wherein the results of the analyzing step indicate that the binding of a molecule has effected modification of the biomolecular target as a result of functional activity, wherein the molecule does not bind to the control biomolecular target and the control biomolecular target is not modified as a result of functional activity.

The present invention is also directed to a method for identifying a modified biomolecular target in a biological sample comprising: contacting a biomolecular target and a control biomolecular target with a biological sample, fractionating the biological sample to obtain a plurality of fractions, analyzing the plurality of fractions by mass spectrometry, determining the mass spectral peak intensity ratio of the control biomolecular target to the biomolecular target for the plurality of fractions, identifying fractions with changes in the peak intensity ratio which indicate specific binding of a molecule to the biomolecular target, identifying within the fractions with changes in the peak intensity ratio one or more additional peaks, comparing the molecular masses of the one or more additional peaks with calculated molecular masses of one or more putative modified biomolecular targets wherein a molecular mass match between a member of the one or more additional peaks and a putative modified biomolecular target identifies the modified biomolecular target.

### BRIEF DESCRIPTION OF THE DRAWINGS

FIG. 1A shows the sequence and proposed secondary structure for E. coli 16S A-site (16S) and control 16S A-site (16SC) synthetic constructs. Base numbering is in reference to full length E. coli 16S RNA. The anchor icon on 16SC refers to the neutral mass tag (described in Example 3).

FIG. 1B is a typical mass spectrum of 16S and 16SC. The peak m/z values represent the monoisotopic species. The asterisk denotes the presence of a synthetic impurity—16SC RNA without the neutral mass tag.

FIG. 2A is a chromatogram of HPLC reversed-phase fractionation with UV absorption detection at 254 nm.

FIG. 2B is a plot of the peak intensity ratio of 16SC/16S vs time with a baseline peak intensity ratio of approximately 0.3 based on the data shown in FIG. 1B.

FIG. 3A is a mass spectrum showing paromomycin (PM) binding specifically to 16S RNA. The peak m/z values represent the monoisotopic species.

FIG. 3B is a mass spectrum showing a species with a mass of ˜327.25 Da binding non-specifically to both 16S and 16SC RNA. The peak m/z values represent the monoisotopic species.

FIG. 4 is a mass spectrum showing the binding species in fraction 146. The peak m/z values represent the monoisotopic species. Two species were observed to bind to 16S RNA: paromomycin (PM) and another species with a molecular weight of 818.35. The percentages shown are relative to the 16S RNA or the 16SC RNA targets respectively. The insert is a magnification of the non-covalent complex (16S RNA and the 818.35 species) showing the isotope envelope of the 5⁻ charge state.

FIG. 5 is a positive mode mass spectrum showing the species observed in fraction 146.

FIG. 6 is a mass spectrum of the results of an assay of a plant extract fraction (PP0007 well 79) indicating the presence of a functional RNase activity which results in cleavage of the 16S RNA A-site construct.

FIG. 7 is a mass spectrum of the results of an assay of a plant extract fraction (PP0013 well 89) indicating the presence of a functional RNase activity which results in cleavage of the 16S RNA A-site construct.

FIG. 8 is a mass spectrum of the results of an assay of a plant extract fraction (PP0031 well 13) indicating the presence of a functional RNase activity which results in cleavage of the 16S RNA A-site construct.

### DESCRIPTION OF EMBODIMENTS

In one embodiment of the present invention, a molecule with functional activity towards a biomolecular target in a biological sample is identified by contacting a biomolecular target and a control biomolecular target with a biological sample, fractionating the biological sample to obtain a plurality of fractions, and analyzing one or more members of the plurality of fractions by mass spectrometry, wherein the results of the analyzing step indicate that the binding of a molecule has effected modification of the biomolecular target as a result of functional activity, wherein said molecule does not bind to the control biomolecular target and the control biomolecular target is not modified as a result of functional activity.

Another embodiment of the present invention is a method for identifying a modified biomolecular target in a biological sample comprising: contacting a biomolecular target and a control biomolecular target with a biological sample, fractionating the biological sample to obtain a plurality of fractions, analyzing the plurality of fractions by mass spectrometry, determining the mass spectral peak intensity ratio of the control biomolecular target to the biomolecular target for the plurality of fractions, identifying fractions with changes in the peak intensity ratio which indicate specific binding of a molecule to the biomolecular target, identifying within the fractions with changes in the peak intensity ratio one or more additional peaks, comparing the molecular masses of the one or more additional peaks with calculated molecular masses of one or more putative modified biomolecular targets wherein a molecular mass match between a member of the one or more additional peaks and a putative modified biomolecular target identifies the modified biomolecular target.

As defined herein, “functional activity” refers to chemical or biochemical action of a chemical or biochemical entity on a biomolecular target which effects a defined change in the covalent structure of the biomolecular target. In some embodiments, the functional activity may be an activity such as protease, nuclease, reductase, kinase, phosphatase or transferase activity.

In some embodiments, the biomolecular target comprises nucleic acid or protein. The nucleic acid can be DNA or RNA either of which comprise a structured region. As defined herein, a “structured region” is a region exhibiting a defined secondary, tertiary, or quaternary structure.

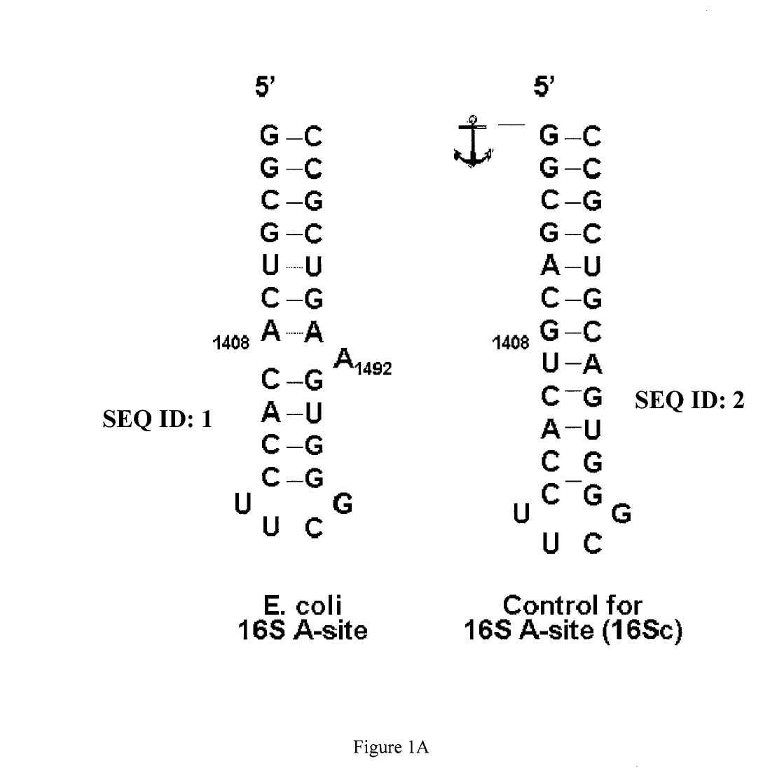

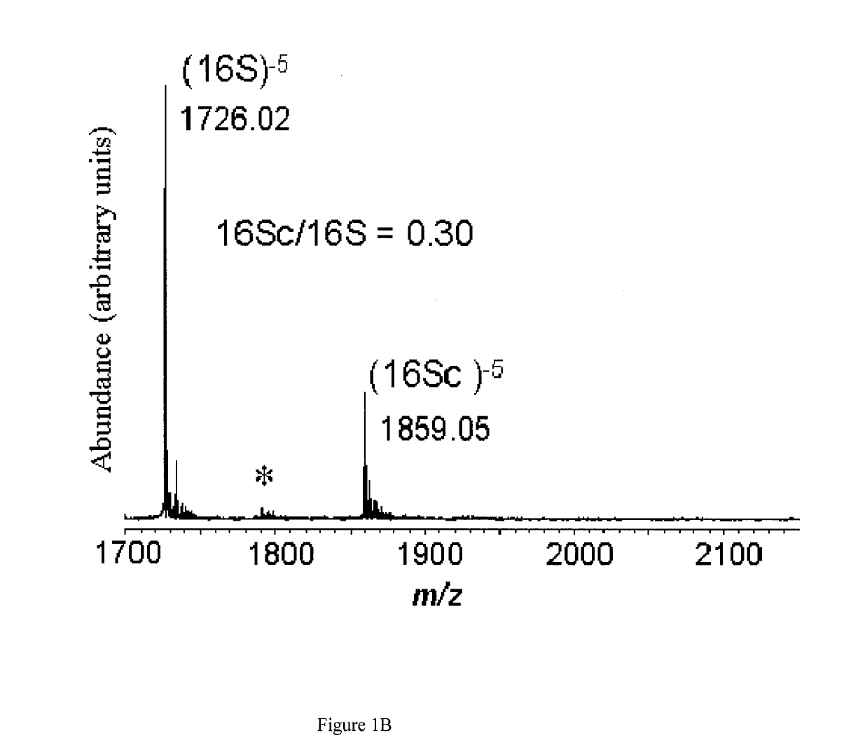

In some embodiments, the biomolecular target is an RNA construct which comprises a structured region and the corresponding control biomolecular target comprises an RNA construct very similar in nature to the RNA construct with the exception that the structured region is absent. For example, as shown in FIG. 1A, the control 16S A-site was created which is highly similar to the 16S A-site with the exception that the structured region (bulge at residues corresponding to positions 1408 and 1492 of E. coli 16S RNA) is eliminated. It is advantageous to design a control biomolecular target similar to the biomolecular target for the sake of confidence of interpretations of differential binding of molecules.

In some embodiments, the mass spectrometric analysis is carried out by a means that preserves the non-covalent interactions between the biomolecular target and the binding molecule. For example Electrospray (ESI) mass spectrometry provides a useful means for preserving such non-covalent interactions. FTICR mass spectrometry provides the necessary sensitivity to characterize the molecular weight of the binding species.

In some embodiments, the functional activity responsible for modification of the biomolecular target may arise from any kind of biomolecule. Classes of small molecules include but are not limited to: carbohydrates, aminoglycosides, macrolides and other natural products or metabolites. Other classes of biomolecules include proteins (including enzymes), complex carbohydrates and lipids. Functional activity may arise due to contact of a combination of biomolecules. For example, binding of a small molecule to a biomolecular target may induce a change in the three dimensional structure of the biomolecule which is then recognized by an enzyme which subsequently modifies the biomolecular target.

The functional activity may involve chemical or enzymatic cleavage of the biomolecular target, such as hydrolysis or addition or removal of a biochemical moiety such as a phosphate group, for example. As defined herein, a biochemical moiety is any chemical group that is found attached to common classes of biomolecules such as proteins, nucleic acids, lipids and carbohydrates.

As used herein, “biological sample” refers to a sample containing one or more biomolecules. Examples of biological samples include but are not limited to: lysates, broths, extracts, assay mixtures and the like.

### EXAMPLES

### Example 1

### Bacterial Strain and Culture Conditions

A dried sample of American Type Culture Collection 14827 (ATCC14827), Streptomyces rimosus sp. paromomycinus, was dissolved and resuspended in 1 ml growth media (24 g corn meal, 11 g Soyabean flour, 4 g NH₄Cl, 15 g CaCO₃, 0.2 g MgSO₄, 50 g D-glucose, 5 g soya oil in 1 liter H₂O). One third of the suspension was used to inoculate 25 ml of sterile media in a 200 ml baffled flask. The culture was incubated in a shaker set at 30° C., 220 rpm for 4 days. Cells and insoluble media components were spun down and supernatant were subject to further analysis.

### Example 2

### Fractionation of Biological Sample Assayed with RNA Constructs

Samples were brought to 0.1% heptafluorobutyric acid (HFBA) by the addition of 1% HFBA. A Gilson HPLC system consisting of four 306 pumps and a Gilson 215 liquid handler was used to perform the separations. Sample injection volume was 3 mL. Separation was carried out using a 250×10 mm Phenomenex Aqua C18 column, with a 50×10 mm guard column. Components were eluted using 0.1% HFBA and a gradient of 0 to 40% Acetonitrile (ACN) at a flow rate of 3 mL/min. over 45 minutes. 1 mL fractions were collected every 20 seconds and were assayed without further preparation.

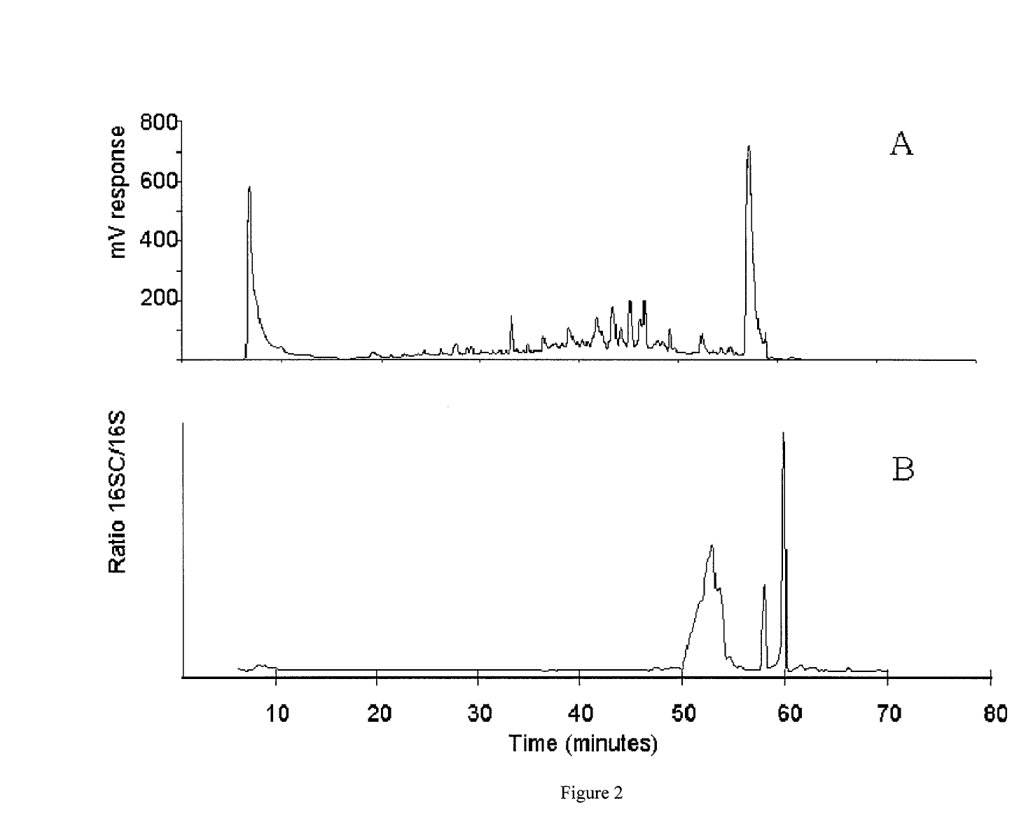

A chromatogram of HPLC reversed-phase fractionation is shown in FIG. 2.

### Example 3

### RNA Constructs

RNA constructs 16S and 16Sc (FIG. 1A) were obtained from Dharmacon Research, Boulder, Colo. The 16Sc construct contains an 18-atom hexaethylene glycol chain attached to the 5′ terminus of the oligonucleotide as supplied by the manufacturer. The addition of this chain results in a net addition of C₁₂H₂₄O₉ P (monoisotopic mass calc 344.1236) to the 16Sc oligonucleotide sequence shown in FIG. 1. The RNA was deprotected according to the manufacturer's directions and ethanol precipitated twice from 1 M ammonium acetate. Paromomycin (MW=615.2963 Da), was obtained from Sigma (St. Louis, Mo.) and ICN (Costa Mesa, Calif.).

The construct shown on the left in FIG. 1A (16S) is the 27-mer synthetic RNA containing the E. coli 16S ribosomal A-site (Purohit, P.; Stern, S. Nature 1994, 370, 659-662). Two positions in the E. coli 16S RNA are noted numerically as 1408 and 1492, referring to the location of these residues in the intact 16S rRNA. These positions have been previously shown to be critical for aminoglycoside recognition and binding (Griffey, R. H.; Hofstadler, S. A.; Sannes-Lowery, K. A.; Ecker, D. J.; Crooke, S. T. Proc. Natl. Acad. Sci. U. S. A. 1999, 96, 10129-10133; Fourmy, D.; Recht, M. I.; Blanchard, S. C.; Puglisi, J. D. Science (Washington, D.C.) 1996, 274, 1367-1371).

The construct shown on the right in FIG. 1A is the 16S control construct (16Sc). It was created by inserting an additional nucleotide (U) at position 1409, replacing the U at position 1406 with an A, and changing the A's at positions 1408 and 1495 to a G and a C respectively. A linker was appended to the 5′ terminus of the control sequence to increase the mass difference between the two constructs and minimize the potential for mass ambiguities (Hofstadler, S. A.; Sannes-Lowery, K. A.; Crooke, S. T.; Ecker, D. J.; Sasmor, H.; Manalili, S.; Griffey, R. H. Anal. Chem. 1999, 71, 3436-3440).

### Example 4

### Mass Spectrometry

Mass spectrometry was performed on a modified Bruker Daltonics (Billerica, Mass.) Apex II 70e electrospray ionization Fourier transform ion cyclotron resonance mass spectrometer equipped with an actively shielded 7 tesla superconducting magnet. Experiments were performed with the source at room temperature, the skimmer potential was held at 0 volts, the capillary exit potential was −126 volts, and other experimental parameters were as described in detail elsewhere (Sannes-Lowery, K. A.; Drader, J. J.; Griffey, R. H.; Hofstadler, S. A. TrAC, Trends Anal. Chem. 2000, 19, 481-491). Binding reactions were comprised of 15 μl of a solution containing 2.5 μM 16S and 16Sc in 100 mM NH₄OAc and 33% isopropyl alcohol and 2 μl of the LC fraction in a 96-well microtiter plate. Under these conditions, the concentration of HFBA contributed by the HPLC fraction does not interfere with mass spectrometric analysis. The plates were vortexed briefly, and then incubated for 60 minutes at room temperature prior to analysis. Sample aliquots were injected directly from 96-well microtiter plates using a CTC HTS PAL autosampler (LEAP Technologies, Carrboro, N.C.). 20 FTICR scans from each well were co-added that, along with the overhead associated with the autosampler, resulted in an analysis time of 39 seconds/well or ˜1 hour/96-well plate.

Accurate mass measurements were performed using angiotensin and bradykinin peptides as internal mass standards. These measurements were obtained using a Bruker Apex 9.4 tesla mass spectrometer. The mass accuracy attained using these standards was ≦1 ppm. Samples were infused at 100 μL/hr in 1% formic acid/25% isopropanol.

### Example 5

### Investigation of a Bacterial Broth Extract for Identification of Molecules that Bind to the 16S RNA A-Site

An example of an embodiment of the present invention is illustrated by a model system comprising a bacterial broth extract from cultures of Streptomyces sp. (which are known to produce aminoglycoside antibiotics) and the E. coli 16S RNA A-site construct described in Example 3.

As is typical of ESI-MS spectra of oligonucleotides, low levels of Na+, K+, or NH4+ adducts are observed on both constructs (FIG. 1B). Several low abundance species are evident between the (16S)-5 and (16Sc)-5 species (see asterisk in FIG. 1B) corresponding to an “untagged” synthetic impurity of the 16Sc construct present at ˜10.0% relative to the tagged 16Sc construct.

The presence of both a specific (16S) and non-specific (16Sc) RNA construct enables the simultaneous determination of binding specificity between target and ligand. In the absence of ligand, the initial peak intensity ratio of 16Sc/16S was determined to be 0.3 as shown in the control spectrum shown in FIG. 1B. The peak intensity ration of 16Sc/16S should remain constant in the absence of ligand binding activity. If a ligand specifically binds the 16S RNA target, the peak intensity ratio of 16Sc/16S will increase, while a ligand that binds non-specifically to both targets will not cause a significant change in the peak intensity ratio of 16Sc/16S. Thus, monitoring the 16Sc/16S peak intensity ratio is a convenient metric for characterizing binding specificity.

The presence of specific and non-specific RNA binding species in a bacterial fermentation broth from S. rimus sp. paromomycinus was determined. Under these growth conditions the broth is expected to contain paromomycin. The UV chromatogram obtained during fractionation of S. rimus sp. Paromomycinus broth is shown in FIG. 2A. The UV trace shows numerous components eluting throughout the 45-minute gradient of 0 to 40% acetonitrile. In a separate experiment, as a control, a sample of commercially available paromomycin was observed to elute between 52 and 53 minutes under these conditions.

The peak intensity ratio of 16Sc/16S RNA from MASS analysis of all 135 HPLC fractions was plotted as a function of elution time and is shown in FIG. 2B. The most significant changes in the peak intensity ratio were observed between ˜50 and ˜60 minutes. The rather broad peak between ˜50 and ˜55 minutes is consistent with the elution time observed for paromomycin. The high concentration of paromomycin present in the sample resulted in a significantly broader peak compared to the paromomycin standard.

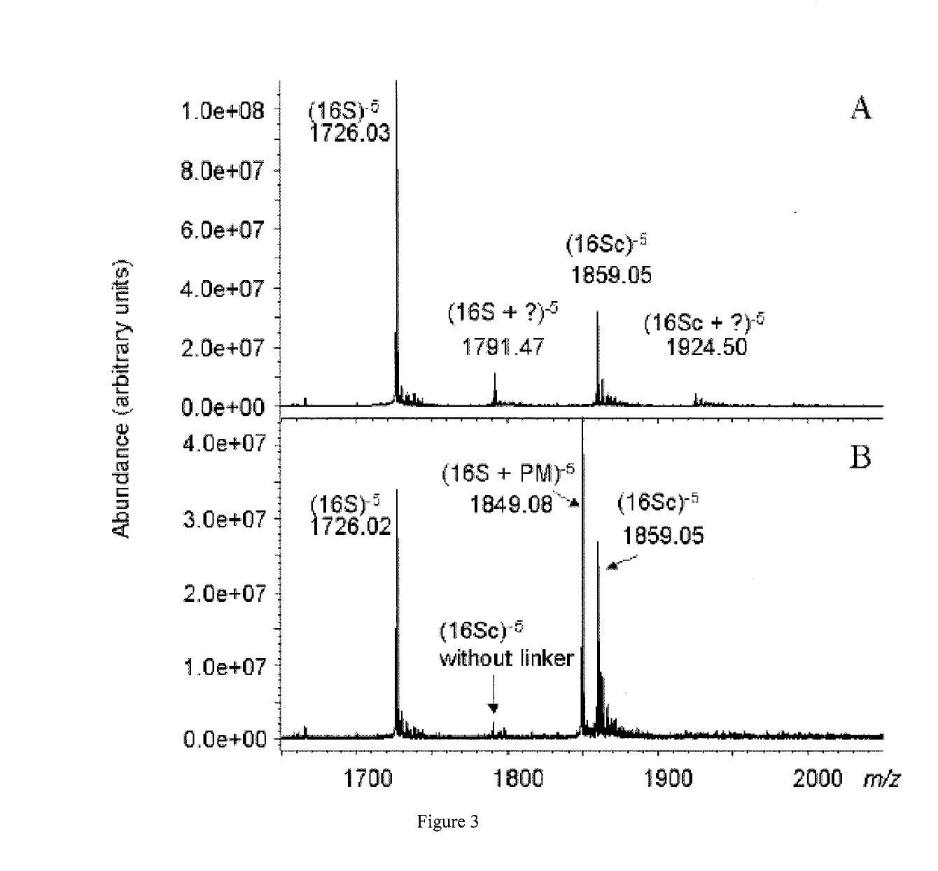

FIG. 3A shows the spectrum obtained for fraction 90 (˜36 minutes). In addition to the 5⁻ charge states of the 16S and 16Sc RNA, two other peaks were observed in the mass spectrum at m/z values of 1791.47 and 1924.50 (monoisotopic m/z values). These peaks represent non-covalent complexes comprised of an unknown compound and the 16S and 16Sc RNA targets respectively. The m/z difference of 65.45 was observed for the 5⁻ charge state complex in each case, and represents a mass of 327.25 Daltons for this species. Since the peak intensity ratio of 16Sc/16S did not change appreciably, it can be concluded that this compound does not bind the 16S target with a significant degree of specificity over the 16Sc construct.

The screening results for fraction #131 (˜49.5 minutes) are shown in FIG. 3B. In this spectrum, the 5⁻ charge state of a species at m/z 1849.08 is observed corresponding to a mass difference of 615.30 Da relative to the 16S target. Both the elution time and molecular weight are consistent with paromomycin (monoisotopic mass (calculated)=615.30). As confirmed below, this peak represents the non-covalent complex between the 16S RNA target and naturally synthesized paromomycin present in the bacterial broth. (The peak broadening, discussed above, results in low levels of paromomycin eluting earlier than the standard, and represents the beginning of a peak with a retention time of ˜52 to 53 minutes). Importantly, examination of the spectrum in FIG. 3B does not indicate a peak corresponding to paromomycin binding to the 16Sc RNA (the peak would be expected at an m/z of 1982.11); an indication of the specificity of paromomycin for the 16S target RNA over that of the 16Sc RNA. This specificity is also indicated by examination of the peak intensity ratio of 16Sc/16S RNA. In the spectrum shown in FIG. 3B, the peak intensity ratio was calculated to be 0.79.

In subsequent fractions, the peak intensity ratio of 16Sc/16S increased as higher concentrations of paromomycin eluted. At sufficiently high paromomycin concentrations the 16S target is completely converted to the 16S-paromomycin noncovalent complex and non-specific complexes between the 16Sc RNA and paromomycin are also observed, albeit at lower abundance (data not shown). In addition, during the peak of the paromomycin elution (e.g. fraction 138), masses consistent with one to four paromomycin molecules binding to 16S and 16Sc RNA were observed (data not shown).

Under conditions when a very high-affinity ligand is present at a high concentration relative to the target concentration, the 16Sc/16S peak intensity ratio may not be as informative because the binding experiment is being carried out under conditions in which the ligand concentration may be higher than the non-specific binding constant of the ligand to the 16Sc RNA. In such instances, one can either dilute the fractions that result in complete binding of the target, increase the target concentration, or perform a 2D separation of the fractions prior to re-screening. In any event, it is most prudent to re-screen fractions containing high concentrations of high affinity ligands as derivatives and/or isoforms of such ligands may not be chromatographically resolved from the primary binding species. This point is illustrated below with a thorough analysis of fraction 146.

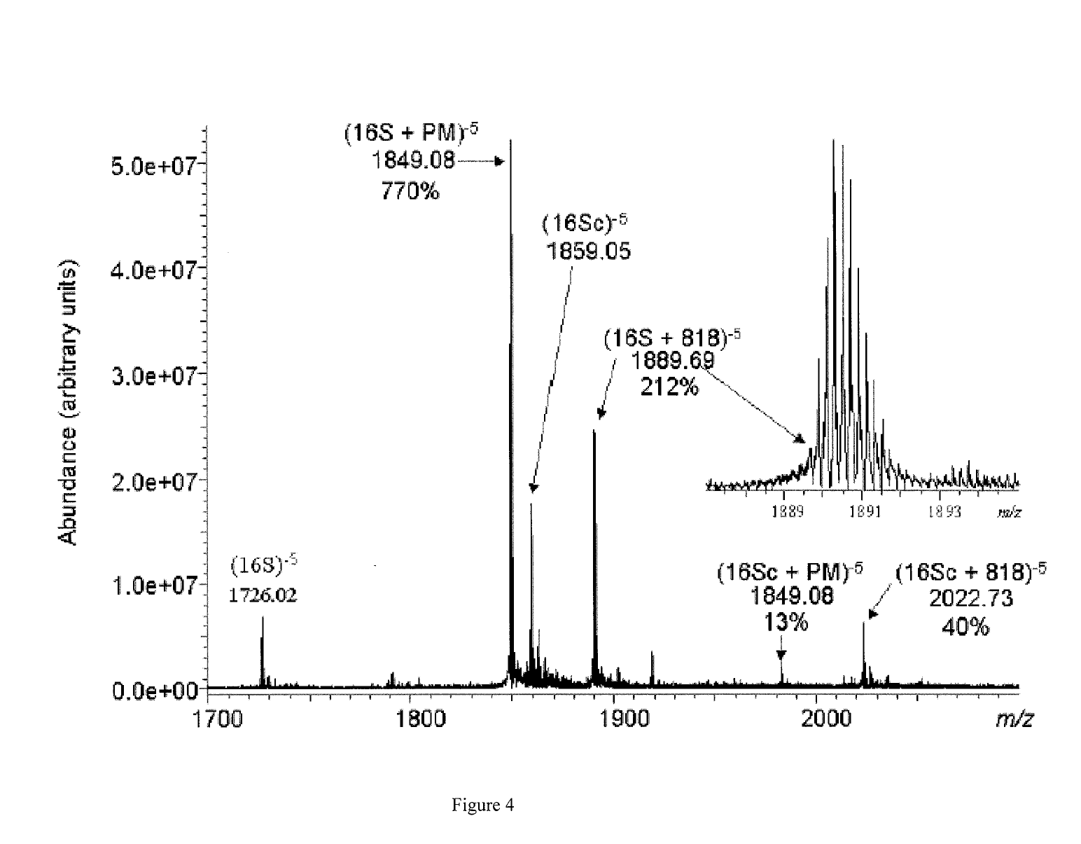

MASS analysis of fraction 146 (˜54.7 minutes) is shown in FIG. 4. Non-covalent complexes between the 16S RNA target and two species are apparent at m/z 1849.08 and 1889.69. The peak at m/z 1849.08 was putatively assigned as the 16S-paromomycin noncovalent complex. The peak at m/z 1889.69 represents a different molecule with a mass of 818.35 Daltons complexed with the 16S RNA target. This new species is also observed at lower abundance as a noncovalent complex with the 16Sc RNA at m/z 2022.73. The abundance of the specific complex of 16S+paromomycin compared to 16S was calculated to be 770% (FIG. 4), while the abundance of the non-specific complex of 16Sc+paromomycin was found to be 13%. These results indicate that paromomycin binds approximately 59-fold more specifically to the 16S target than it does to the 16Sc construct. A similar comparison of the new species indicates that it binds with approximately a 5-fold specificity to the 16S RNA target over the 16Sc RNA construct. Similarly, these results indicate that paromomycin binds to the 16S RNA target with approximately 11-fold more specificity than this new molecule (59-fold vs. 5-fold).

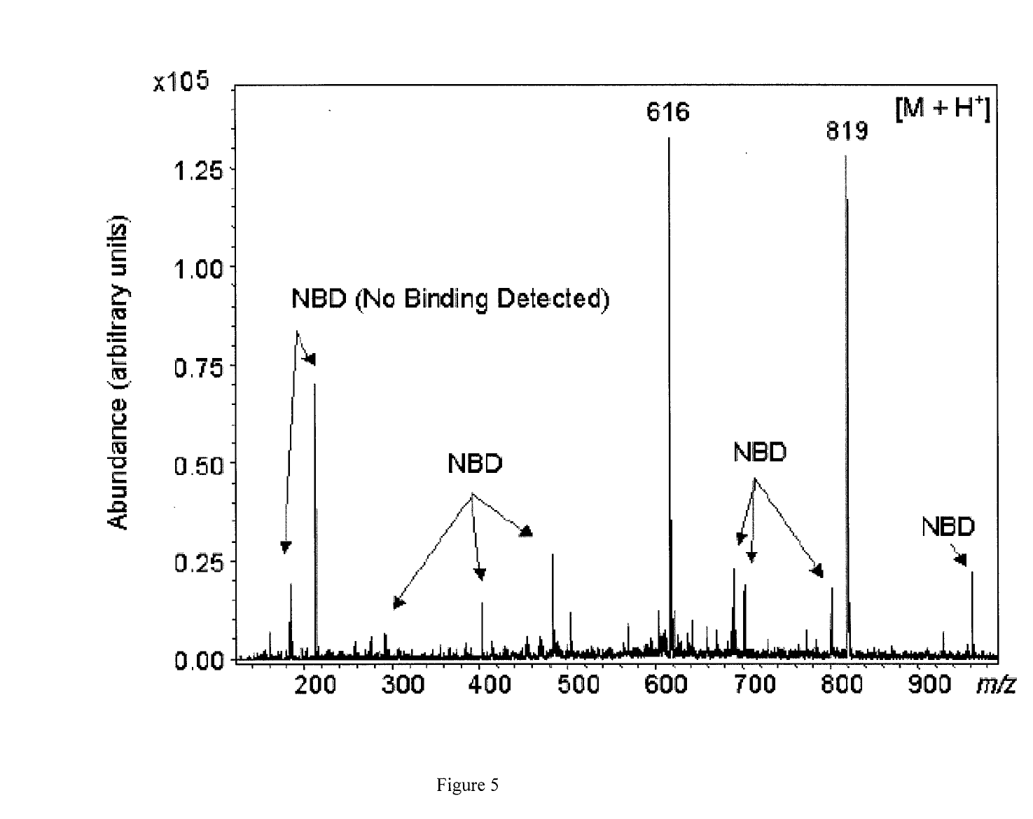

FIG. 5 shows a positive mode mass spectrum of fraction 146. The components observed in fraction 146 include the (M+H⁺) species of what is shown below to be paromomycin (m/z 616), the (M+H⁺) species of the 818 compound (m/z 819), and several other species which were present in the fraction, but which did not bind to the 16S or 16Sc RNA (FIG. 5). Paromomycin and the unknown compound were the most abundant peaks detected, and, assuming that their ionization efficiencies are comparable, were likely present at similar concentrations. Fractions in the vicinity of fraction 146 are particularly interesting as in addition to containing paromomycin; they also contain the 818 species that, based on chromatographic retention and 16S binding, is most likely a paromomycin derivative.

An accurate mass measurement of the presumed paromomycin ((M+H⁺)˜616) from fraction 146 was performed on a 9.4 tesla FTICR mass spectrometer. Mass accuracy with sub-ppm mass measurement error was achieved using internal mass standards. The mass was measured to be 616.3035±0.0006 (C₂₃H₄₆O₁₄N₅ calc 616.3036). MS/MS fragmentation of this species gave daughter ions consistent with those of paromomycin (data not shown). The MS/MS spectrum produced from isolation and fragmentation of the novel species generated a daughter ion at m/z 616. Further fragmentation of this daughter ion resulted in daughter ions consistent with those of paromomycin (Curcuruto, O.; Kennedy, G.; Hamdan, M. Org. Mass Spectrom. 1994, 29, 547-552; DeJohngh, D. C.; Hribar, J. D.; Hanessian, S.; Woo, P. W. K. J. Am. Chem. Soc. 1967, 89, 3364-3365; Goolsby, B. J.; Brodbelt, J. S. J. Mass Spectrom. 2000, 35, 1011-1024).

These data suggest that the 819 species is composed of a core paromomycin moiety that has been modified on one or more of its rings.

### Example 6

### Identification of Functional Activity Towards the 16S RNA A-Site

An additional observation was made from the experiment discussed in Example 5. Fractions 156 (˜58 Minutes) through 162 (˜60 Minutes) demonstrated relatively sharp peaks in the peak intensity ratio plot of 16Sc/16S (FIG. 2B). A number of peaks were observed with masses from ˜650 Da to ˜7700 Da which were not evident in the direct ESI-MS analysis of the fractions. These masses were compared with potential degradation products of the 16S and 16Sc targets and were found to match these expected products and correspond to RNA oligonucleotides in the 2-mer to 24-mer size range. These data suggest that the constituents of these fractions induced limited hydrolysis of both targets. The 16S RNA appeared to be hydrolyzed to a greater degree than the 16Sc RNA when the levels of each were compared, possible due to the neutral mass tag “cap” on the 5′ end of the 16Sc construct, or the tighter stem structure of the fully Watson-Crick base paired stem.

These data suggest that, in addition to finding small molecules that bind to biomolecular targets of interest, the screening methods described herein can be used to identify natural product fractions with functional activity consistent with activities that include, but are not limited to: nuclease, protease, reductase, kinase, phosphatase, or transferase activities. It is expected that such functional activity may arise from the action of an enzyme, a non-proteinaceous molecule or a combination thereof.

### Example 7

### Screening of Plant Extracts for Fractions Containing Molecules with Functional Activity

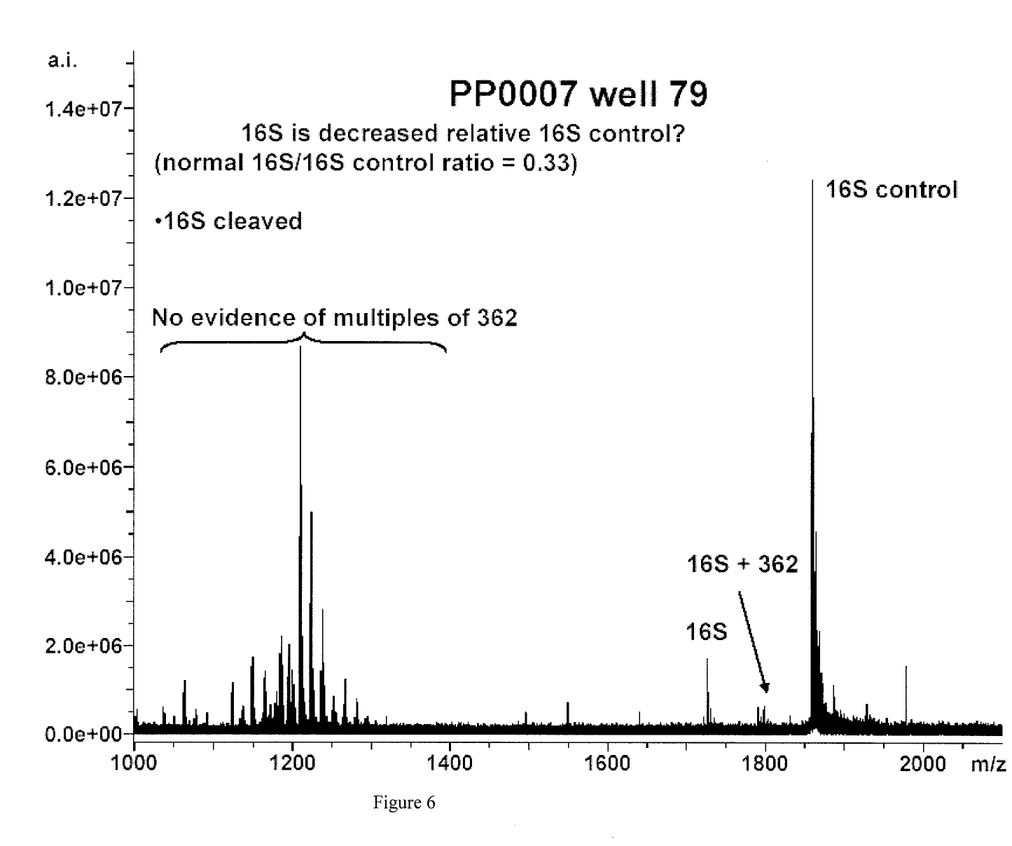

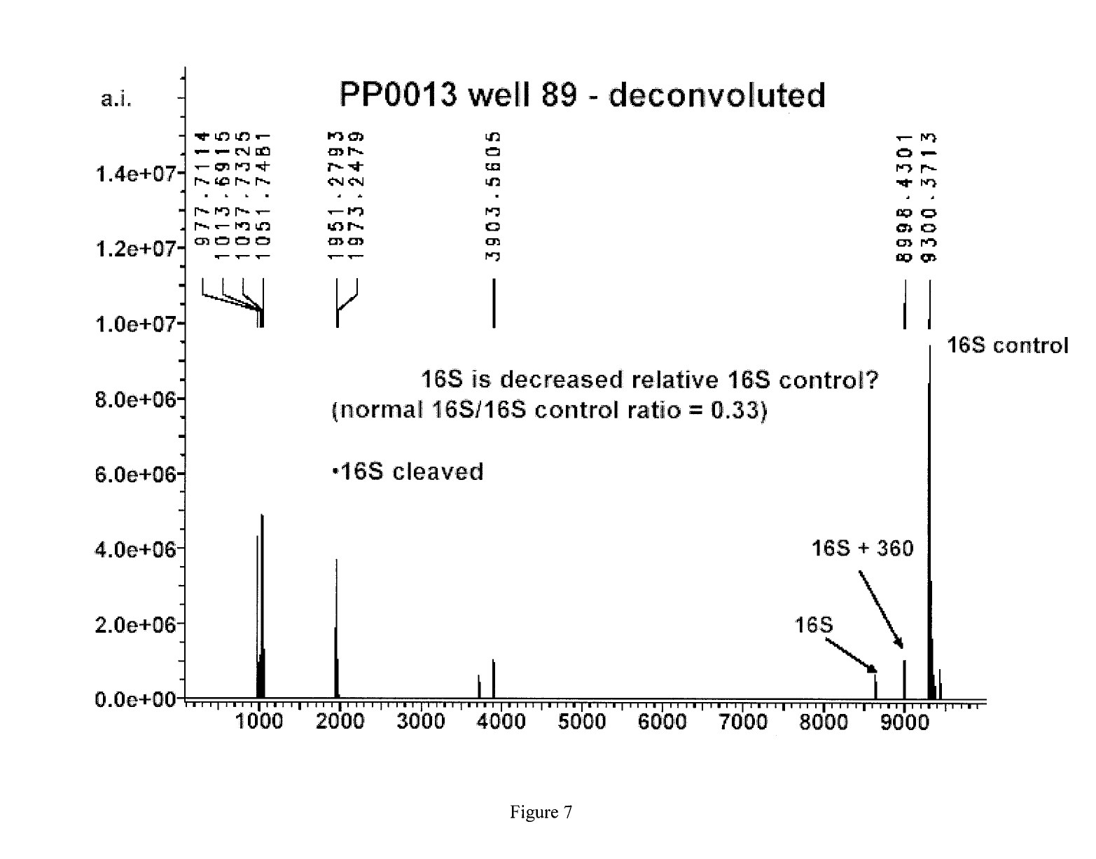

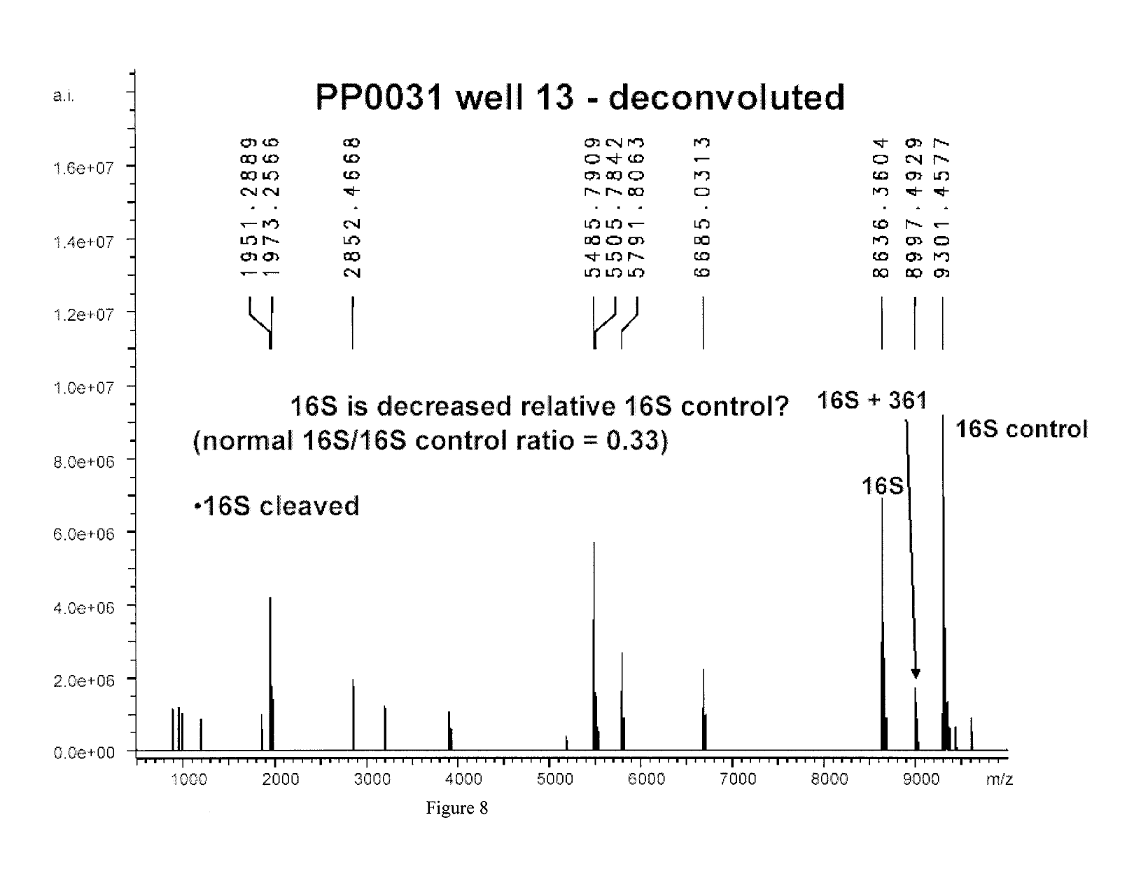

A series of plant extracts were examined by the methods outlined in Examples 1-6 to identify fractions that contain functional activity towards the E. coli 16S A-Site. FIGS. 6, 7 and 8 represent mass spectra of fractions that contain RNase activity which is responsible for the cleavage of the 16S RNA construct.

Various modifications of the invention, in addition to those described herein, will be apparent to those skilled in the art from the foregoing description. Such modifications are also intended to fall within the scope of the appended claims. Each of the patents, applications, printed publications, and other published documents mentioned or referred to in this specification are incorporated herein by reference in their entirety. Those skilled in the art will appreciate that numerous changes and modifications may be made to the embodiments of the invention and that such changes and modifications may be made without departing from the spirit of the invention. It is therefore intended that the appended claims cover all such equivalent variations as fall within the true spirit and scope of the invention.

What is claimed is:

1. A method for identification of a molecule with functional activity towards a biomolecular target in a biological sample comprising:
fractionating a biological sample to obtain a plurality of fractions;
selecting one or more of said fractions;
selecting a biomolecular target;
selecting a control biomolecular target having a modified structured region as compared to said biomolecular target;
adding said biomolecular target and said control biomolecular target to said selected fractions; and
analyzing one or more of said selected fractions by mass spectrometry for mass peaks indicating that the binding of said molecule in said selected fraction has effected modification of said biomolecular target as a result of functional activity and concurrently said molecule did not bind sufficiently to said control biomolecular target such that said control biomolecular target was modified as a result of said functional activity.

2. The method of claim 1 wherein said selected fractions are maintained under non-denaturing conditions.

3. The method of claim 1 wherein said molecule is a small molecule.

4. The method of claim 1 wherein said biomolecular target comprises nucleic acid or protein.

5. The method of claim 1 wherein said bimolecular target comprises an RNA construct having said structured region; and
said control biomolecular target comprises an RNA construct without said structured region.

6. The method of claim 1 wherein said fractionating step comprises reverse-phase chromatography.

7. The method of claim 1 wherein said mass spectrometry comprises ESI-FTICR mass spectrometry.

8. The method of claim 1 wherein said modification of said biomolecular target comprises chemical or enzymatic cleavage.

9. The method of claim 1 wherein said modification of said biomolecular target comprises addition of a biochemical moiety.

10. The method of claim 9 wherein said biochemical moiety comprises a functional group.

11. The method of claim 1 wherein said functional activity comprises nuclease, protease, reductase, kinase, phosphatase, or transferase activity.

12. The method of claim 1 wherein said molecule comprises an aminoglycoside, a macrolide, or an enzyme.

13. The method of claim 1 wherein said binding of said molecule effects recruitment of an enzyme which then effects said modification of said biomolecular target.

14. The method of claim 1 wherein said biological sample comprises a lysate, an extract, a broth, or a mixture of natural products.

15. A method for identifying a modified biomolecular target in a biological sample comprising:
fractionating a biological sample to obtain a plurality of fractions;
selecting one or more of said fractions;
selecting a biomolecular target;
selecting a control biomolecular target having a modified structured region as compared to said biomolecular target;
mixing said said biomolecular target and said control biomolecular to form a target mixture;
determining the mass spectral peak intensity ratio of said control biomolecular target to said biomolecular target in said target mixture;
adding an aliquot of said target mixture to said selected fractions;
determining the mass spectral peak intensity ratio of said control biomolecular target to said biomolecular target in said selected fractions;
identifying fractions with changes in the peak intensity ratio which indicate specific binding of a molecule to said biomolecular target;
identifying within said fractions with changes in the peak intensity ratio, one or more additional peaks; and
comparing the molecular masses of said one or more additional peaks with calculated molecular masses of one or more putative modified biomolecular targets wherein a molecular mass match between a member of said one or more additional peaks and a putative modified biomolecular target identifies the modified biomolecular target.

16. The method of claim 15 wherein said selected fractions are maintained under non-denaturing conditions.

17. The method of claim 15 wherein said molecule is a small molecule.

18. The method of claim 15 wherein said biomolecular target comprises nucleic acid or protein.

19. The method of claim 15 wherein said bimolecular target comprises an RNA construct having a structured region; and
said control biomolecular target comprises an RNA construct without said structured region.

20. The method of claim 15 wherein said fractionating step comprises reverse-phase chromatography.

21. The method of claim 15 wherein said mass spectrometry comprises ESI-FTICR mass spectrometry.

22. The method of claim 15 wherein said molecule comprises an aminoglycoside, a macrolide or an enzyme.

23. The method of claim 15 wherein the binding of said molecule effects recruitment of an enzyme which then effects said modification of said biomolecular target.

24. The method of claim 15 wherein said biological sample comprises a lysate, an extract, a broth, or a mixture of natural products.
